(19) United States
(12) Patent Application Publication
(10) Pub. No.: US 2006/0278236 A1
(43) Pub. Date: Dec. 14, 2006

(54) Universal unisex safety condom

(76) Inventor: Daniel Resnic, Beverly Hills, CA (US)

Correspondence Address: ERIC HANSCOM, 7395 PORTAGE WAY, CARLSBAD, CA, 92011, US

(21) Appl. No.: 11/505,549

(22) Filed: Aug. 18, 2006

Related U.S. Application Data
(63) Continuation-in-part of application No. 10/681,076, filed on Oct. 7, 2003.
(60) Provisional application No. 60/416,960, filed on Oct. 7, 2002.

(51) Int. Cl. A61F 6/04

(52) U.S. Cl. 128/844

(57) ABSTRACT

This invention relates to the field of contraception and to the control of venereal disease transmission, including AIDS and HIV infection. The invention is a method of making an improved condom which can be used by either a male or by a female partner and can be inserted into a sexual orifice without first rolling it over an erect penis.

### CROSS REFERENCE TO RELATED APPLICATION

Provisional Appl. No. 60/416,960 filed Oct. 7, 2002 and to Ser. No. 10/681,076, a Utility Patent Application filed on Oct. 7, 2003.

### FEDERALLY SPONSORED RESEARCH

Not Applicable

### SEQUENCE LISTING OR PROGRAM

Not Applicable

### BACKGROUND OF THE INVENTION

1. Field of the Invention

This invention relates to the field of contraception and to the control of venereal disease transmission, including AIDS and HIV infection. In particular, this invention relates to an improved condom and condom assembly for use by either a male or by a female partner.

2. Background of the Invention

The art of the present invention resolves many important issues that have prevented the traditional, thin-walled condom from being used as effectively and extensively as needed in the HIV/AIDS crisis. It may help break the usage barrier surrounding contraception efforts around the world.

Amid the myriad forms and methods of contraception the condom has recently become the subject of intense medical and commercial interest. The complications associated with intrauterine devices and birth control pills have mitigated against their use in most cases. Moreover, the appearance of the HIV virus and possible impending AIDS have created an atmosphere of caution and prudence in which sexually active adults must consider protecting themselves from infection by HIV virus and other venereal diseases. Aside from complete sexual abstinence it is a generally accepted fact that the condom provides the best protection from venereal disease and HIV virus.

Since the onset of AIDS in 1981, a time of dreaded venereal diseases and sexual freedom, people have been increasingly encouraged by the medical profession, as well as by governmental and other health organizations, to use condoms to prevent the spread of venereal diseases. However, condoms are well known to be only partially effective. Leakage and breakage both serve to reduce the effectiveness of condoms as a barrier to contagion and to conception.

As it is generally perceived, a near-universal dissatisfaction with traditional latex condoms marks the history of condom usage. This is a function of impractical design and lack of user-friendliness in prior the art. The traditional condom is clumsy to unwrap, difficult to unroll, creates unwanted interruptions at the time of use, has a significant failure rate and diminishes sensitivity for the partners.

Protection from STD, STI, pregnancy, etc. is a concern of sexual partners of every sexual orientation, nationality or age group. After the discovery of AIDS in 1981, gay men in urban areas of America comprised approximately two-thirds of all AIDS cases in the United States, Europe, and Latin America. The percentages, however, rapidly changed due to increasing numbers of heterosexual women becoming infected through sexual contact with infected men. It is expected that most new infections will occur in women according to the World Health Organization (WHO).

For this reason, therefore, unlike most prior art, the teachings of this invention will hereinafter reference descriptions of sexual partners and terms, which identify anatomical features and specific roles by men, and women of heterosexual and/or homosexual orientations as follows;

| | |
|---|---|
| (i) | Male active partner (penetrating) |
| (ii) | Female passive partner (receiving) |
| (iii) | Male passive partner (receiving) |
| (iv) | Vaginal orifice |
| (v) | Female condom |
| (vi) | Male condom |
| (vii) | Anal orifice |
| (viii) | Receptive condom |

Devices that prevent unwanted conception and/or exchange of bodily fluids during sexual intercourse have been known in the art for many years. Female contraceptives are generally divided into four basic types which include the cervical cap; devices that rely on chemical spermicides, such as diaphragms, sponges, foams, jellies, dermal implants, patches and suppositories, oral pharmaceuticals and the intrauterine device. These contraceptives, however, are marked with a number of serious disadvantages.

Although effective in preventing conception, oral contraceptives reportedly have a range of undesirable side effects, including possible carcinogenic affects to the liver and gall bladder. The diaphragm is reliable however it is an awkward and inconvenient contraceptive that easily can interrupt or distract from the act of intercourse.

Spermicidal

contraceptives are inconvenient to use and may be only as much as 80% reliable as a contraceptive. The cervical cap cannot be fitted on some women, and on women who are able to wear it, it may produce discomfort and infection. The intrauterine device has been the subject of much public controversy and has been reported to be unsafe under certain conditions.

There may also be a possibility of danger to women who are susceptible to toxic shock syndrome, since the cervical cap, the intrauterine device and spermicidal implants are all foreign bodies that are left inside the vaginal vault either permanently or for several hours after intercourse.

Various known methods of female contraception do not provide adequate protection from sexually transmitted diseases. Although spermicidal implants may provide some protection, the available female contraceptives do not provide a reliable impermeable barrier to STD and STI.

Unlike female contraceptives, the male condom is the usual type of contraceptive available to men. The thin membrane latex male condom provides a reliable, impervious membrane barrier against the transmission of infections. Additionally, the traditional male condom is always readily available, economical, does not require a doctor's involvement, and does not normally cause any side effects to either partner except for toxic allergic reactions to latex in some users. It is however the most effective protection against STD and STI available to either men or women although the male condom can have a failure rate up to 17%.

The traditional male condom comprises an elongated tubular sheath made of thin, flexible material such as latex, plastic or polyurethane film. The tubular device is closed at the distal end and open at the proximal end in order to provide for the insertion of a penis and subsequently contain ejaculate of seminal fluids. The proximal end includes a peripheral bead which functions as a constricted rim as a means of retaining the device against slippage and to further contain ejaculate. The condom device is placed on the penis of a male partner by rolling, stretch or pulling it onto an erect penis. The thin walled latex condom clings tightly to the penis in order to be kept in place during intercourse.

Condoms as known in the prior art are known to suffer several drawbacks, chiefly in that they interfere with the normal progression of intercourse. Use of the condom requires male arousal first, followed by an interruption to open a condom package and apply the condom to the male member. A major complaint is that they desensitize the act of intercourse for an active male partner. Other disadvantages include slippage, breakage, and leakage. After sexual intercourse is completed, a traditional condom may slip off if the penis continues to soften and is left inside the passive partner, thus causing unwanted leakage of semen and/or infectious matter. In addition, the traditional thin latex male condom requires that the penis be erect in order to initially don the condom. This undesirable interruption causes an untimely distraction of the sex act. Furthermore, the membrane of the condom, no matter how pliant or thin, is often viewed as a barrier to the most intimate tactile sensations of intercourse.

Yet another disadvantage is the loss of sensation experienced by the active male caused by the fact that the traditional male condom must be close-fitting in order to stay in place thus minimizing the natural tactile stimulation. Because of the loss of sensation and inconvenience, it is universally considered that the use of condoms is undesirable and there is often great resistance to their use on a regular basis.

Additionally, most of all prior art is thin-walled construction which, when in place, is intended to cover and conform to the overall shape of an erect penis, however this objective is not possible considering that a penis of larger or smaller length and girth will not fit properly into a standard, generic size condom.

The prior art, both traditional as well as newly designed thin, form-fitting condom devices do not permit adequate sensitivity for a male during sexual intercourse because a form fitting condom moves along with the penis 6 and prevents direct tactile contact and friction between the vaginal or rectal wall of a passive partner and the penis 6 of an active male partner during sexual intercourse.

Normally the conventional thin condom is made of strong, fine rubber, or some type of fine animal skin or a synthetic membrane such as polyurethane. In order to provide an acceptable level of tactile stimulation to the active male wearer, these condoms must be quite thin. In general, it is elastically fitted to the male organ and during coitus remains outstretched and taut. This taut condition can increase the hazard of the condom being torn or bursting during use and can also compromise the integrity of the condom insofar as leakage and slippage are concerned. Traditional methods of manufacturing a contraceptive, prophylactic or condom involve the so-called straight dipping or dip and dry techniques wherein a phallic shaped mandrel, normally of ceramic, metal or glass composition, of predetermined size is, optionally coated with a coagulating agent, and dipped into a bath containing natural rubber latex or a solution of synthetic material such as polyurethane. The mandrel when dipped may be stationary or rotating about its longitudinal axis. The mandrel, when immersed in the latex bath is immersed to a depth sufficient to yield the finished condom of desired length plus an additional distance to allow for rolling the sheath on itself for several turns in order to form a latex ring at the open end of the condom or optionally to a depth coincident with the upper edge of the circumferential groove which would then form the latex ring. After a predetermined period of time the mandrel, covered with a coating of latex, which conforms to the mandrel's shape, is withdrawn.

The present art can be manufactured by casting, injection molding or thermal molding methods that are not presently used within the condom industry for fabricating either male or female condoms. Further there are no condoms presently manufactured using these methods. Such a condom is not prohibitively expensive to manufacture by these methods.

There is currently marketed a female condom consisting of a flexible thin-walled, bag-like liner with a closed end and an open end opposite the closed end. The liner is formed from two sheets of plastic material sealed together at their edges, and a solid ring of relatively stiff plastic material is provided inside the liner at the open end and acts to hold the mouth of the liner open. The female condom has not met with significant market success due the cumbersome method of insertion that incorporates the ring as a means of retaining.

One of the disadvantages of a ‘female’ or receptive condom is that there is a relatively high risk of penetration outside the condom, particularly with repeated contacts. For a female or receptive condom to be an effective barrier to pregnancy and/or the transmission of the AIDS virus or other viruses and microbes, it is essential that, even during repeated contacts, the penis 6 is unable to penetrate the vagina 13 outside the condom. That is why it is advantageous to provide a protective device in which a shield formation at the proximal end 2a comprises a cuff shaped protuberance 2 placed parallel to the receptive orifice 8, 14 and perpendicular to the tubular part of the condom and prevents the proximal end from passing into the receptive orifice of a passive partner.

The universal, unisex condom of the present invention may include but does not require the use of spermicidal chemical or anti-disease medication. Like conventional condoms, the invention can be removed after intercourse, unlike the sponge or diaphragm, which must be left in for six to eight hours to be effective. In addition the preferred material is biocompatible, a definite advantage to a partner who is susceptible to toxic shock syndrome or allergic reactions to chemicals or materials associated with latex devices in the prior art.

From a commercial standpoint, surveys have shown that nearly 50% of condoms purchased are sold to women, although it is clear that those condoms, as known in the prior art, must be worn by the male. Therefore there is an implication that many women are concerned about contraception and disease prevention, and desirous of taking steps toward prevention of both. There is also additional interest among the gay communities for an internal condom for a passive user. However, there is no device known in the prior art that prevents conception and disease, and can be used either by a male or a by a female for intercourse.

There have been numerous proposals of thin walled condoms, which are designed to transfer tactile sensation through the wall of the membrane of the condom device, which consequently minimize and diminish the natural stimulation of sexual intercourse between two partners.

Many attempts have been made to design a condom or condom-like device as a contraceptive/prophylaxis device to be worn on the inside of passive sexual organs of a passive partner and, in addition, a condom or condom-like device as a contraceptive/prophylaxis device to be worn and retained on the outside of a penis.

For examples of such condom devices see U.S. Pat. Nos. 4,004,591; 4,320,752; 4,323,675; 4,354,494; 4,798,600; 4,805,604; 4,867,176; 4,869,723; 4,898,184; 4,945,923; 4,955,392; 4,976,273; 5,069,228; 5,070,890; 5,074,314; 5,094,250; 5,269,320; 5,314,447; 5,370,131; 5,398,699; 5,433,219; 5,454,379; 5,718,236; 5,318,042; 5,515,862; 5,718,236; 5,803,085; 6,223,747; 6,341,607; 899,251. Also, among these devices are included condoms having a disposable applicator or condoms having integrated retaining means, e.g. rings, to secure the condom within the passive sexual organ. A sampling of these various attempts is described below.

U.S. Pat. No. 4,004,591 to Freimark discloses a birth control device, a female condom made of a strong rubber, plastic, or other similar material. This condom has a rigid, ring-like rim that is bent or scalloped. This rim can be a wire. The rim is not adapted to radially extend the open end of this device because this device is a hard, molded, inflexible material. Dimensions of this condom are disclosed as being sufficiently large to accommodate the average width of the penis with some additional clearance space and there-fore would exclude a portion of a population from using it. The primary function of this device is to prevent unwanted pregnancy, however, this device provides no means at the vulva to prevent an exchange between partners of secreted fluids that can contain venereal disease. In addition, this birth control device is intended for wear by females, but includes no means to secure or maintain the device within the vagina. No provision is cited to use the device for anal intercourse.

U.S. Pat. No. 4,323,675 reveals a condom using a harness inside the condom sheath to increase sensation and reliability. The device appears overly complex and generally removed from the natural sensations of intercourse. Consequently this may discourage use and render it ineffective. No provision is cited to use the device for anal intercourse.

U.S. Pat. No. 4,354,494 does provide a restraining strap that fits over the scrotum. However, it is unclear if this strap would remain secure during sexual intercourse and if tension from this strap would cause discomfort to the testicles, whereas the restraining strap of the present invention is designed to provide a highly stretchable, comfortable and secure fit. Also significant, this device does not provide a reservoir tip and therefore unable to provide a comparable level of protection as with the present invention. Accordingly, there is a need in the art for a simple and convenient condom assembly and method of application for a sexually passive partner, which overcomes many of the disadvantages and deficiencies of conventional devices mentioned herein.

U.S. Pat. No. 4,798,600 discloses a female condom consisting of two condoms joined together by a ring, a male portion with an open end and a female portion with an open and a closed end. The device is designed to operate with the male portion moving inside the anchored female portion when in use. While this device claims a high degree of stimulation of the penis it fails to provide an adequate means to secure the condom to the penis leaving the orifice of a passive partner at risk of possible entry by the penis or by semen during coitus. If the penis is withdrawn too far during coitus it can create an undesired interruption of the sex act and again presents a possible risk that the penis may enter the orifice of the passive partner between the exterior wall of the female portion at the ring and the vagina or anus of the passive partner without the knowledge of either partner. The invention clearly demonstrates a conscientious recognition for use by a passive male partner, however it provides only one means to retain the device, that being exclusively with the passive partner in the form of a multiplicity of ridges along the exterior surface of the insertable, female portion of the device. The device has no reservoir to help protect the passive partner from potential backflow of semen.

U.S. Pat. No. 4,805,604 discloses a receptive condom device adapted to be received by a passive sexual organ prior to intercourse. The device consists of a thin elastic membrane sheath having a closed end, a supporting ring structure at its open end, and an insertion tube adapted to deliver the condom into a passive sexual organ prior to intercourse. A charge of compressed air, in communication with the inside of the condom, forces the condom out of the tube end into the passive sexual organ, closed end first.

The collapsed charge and insertion tube are then removed, leaving the condom in place. While the condom device is somewhat functional for its intended use, the structure appears too flaccid to maintain its' shape within the vaginal cavity. Consequently the device risks unintentional, partial or complete removal form the vagina. Also, the method of application described could be significantly improved.

U.S. Pat. No. 4,867,176 describes a female condom and a disposable tampon-like applicator to facilitate vaginal insertion. The condom is inserted into the vagina using the applicator fitted with a broad tip portion, which is received in the closed distal end of the condom. The applicator is then removed, leaving the condom in place. A disadvantage of the described device is that the applicator is cumbersome and might inadvertently tear the condom during insertion into the vagina or it could pull the thin condom out from the vagina during removal of the applicator.

U.S. Pat. No. 4,869,723 describes a condom designed specifically with intent to provide enhanced male stimulation and feeling at the distal end of the penis for the benefit of an active male user. Harmon compares it to be quite similar in appearance to a conventional condom. While this device provides a means for partial sliding of the penis and an enlarged major section to help facilitate retention within the orifice of a passive partner, it is not constructed of a rigid or semi-rigid material which can fail the means for retention to maintain its larger dimension and shape when in use. The device has no reservoir to help protect the passive partner from potential backflow of semen and consequential contamination. Additionally the device does not provide a means to facilitate insertion into the orifice of a passive partner.

U.S. Pat. No. 4,898,184 to Skurkovich et al. describes a female condom having a number of shield portions connected to a genital portion. FIGS. 1 and 2 show the Skurkovich device, in which their numbering system has been preserved and is used here in parentheses. According to their description, the female condom has an upper portion or apron (20) covering the lower abdomen and pubic area, a vaginal portion (22) and thigh portions (24) and (26). Thin belt portions (28), (30), (32) and (34) are integrally connected to the apron and thigh portions for attachment. Alternatively, the various portions may be attached to the skin by a narrow strip of glue-like material. FIG. 1 shows clearly that thigh portion (24) covers only the front of the thighs, leaving the rear of the inner thighs, part of the perineum and the anal area exposed. FIG. 2, (in which the triangular shape is somewhat enigmatic in its relation to thigh portions (24) and (26), and vaginal portion (22) seems to have vanished) shows clearly that upper portion (20) stops short of straps (30), (34), and therefore fails to give protection to the bottom of the groin and the front of the inner thigh. The weak glue, such as the 3M yellow paper glue, mentioned as an alternative method of attachment would be insufficient to hold the device in place without the straps. No provisions are described that prevent seepage of body fluids beneath the various portions of the device.

U.S. Pat. Nos. 4,945,923 and 4,976,273 describe a vaginal sheath having flexible rings at both the top open and bottom closed ends. The bottom ring portion of the sheath is inserted into the vagina for positioning against the cervix like a diaphragm. The disadvantage of the described devices is that they are awkward and difficult to fit. It is also considered that such devices are difficult to correctly insert into the vagina. See, for example, E. H. in “Mirabella,” August 1992, page 70 and F. Vrazo in “New York Daily News,” Jan. 22, 1990.

U.S. Pat. No. 4,955,392 discloses a thin-walled, adhesive fitted male condom. Adhesive is positioned on the body side of a circular collar shield, however adhesion is not effective where the adhesive aligns with the pubic hair region. In addition it cannot provide adequate stretch for comfort and fit of a penis of substantially larger length and/or girth.

U.S. Pat. No. 5,069,228 describes a male condom with a small shield attached to the user by a peripheral ring of adhesive. The adhesive described is a copolymer of an acrylic ester and acrylic acid, which is a water resistant glue, painlessly removable from the skin. However, the device described is flawed. The adhesive perimeter is unable to adhere to the body to form a leak proof seal because, like U.S. Pat. No. 4,955,392, the location of the adhesive is structured to fit over the pubic hair, thus compromising the device and making the adhesive shield ineffective.

For example, U.S. Pat. No. 5,070,890 and U.S. Pat. No. 5,314,447 include a scrotal sac in each embodiment, but the sac opening has the disadvantages of not being substantially wider than the shaft and of not being made of a pliant construction, thus hindering its' application to the scrotal sac. Since condom usage is often dependent on convenience and comfort, such a limitation could easily discourage usage, possibly during a sexual encounter where usage is necessary to prevent disease and/or pregnancy.

U.S. Pat. No. 5,074,314 describes a protective sheath for use in a bodily orifice containing a inner solid retention element at the closed bottom end for retaining or securing the sheath within that orifice. The disadvantage of the described device is that the retention element may cause physical discomfort for the wearer during sexual intercourse. Additionally, the protective sheath does not include an applicator to facilitate proper alignment of the retention means within the vagina. No provision is cited to use the device for anal intercourse.

Many attempts have been made to develop a female condom, but none has yet provided reliable, user-friendly contraception and effective protection from sexually transmitted diseases. U.S. Pat. No. 5,094,250 to Hessel describes a device, having a flexible tube with an open end and a closed end, a first ring for positioning the closed end of the device at the bottom of the vagina, and a second ring for preventing the open end of the tube from entering the vagina. This device, besides being difficult to keep in place during intercourse, does not protect against seepage around the second ring. It also fails to give protection against diseases transmitted by body fluids contacting the perineum or anal region. Further, no provision is cited to use the device for anal intercourse.

U.S. Pat. No. 5,269,320 to Hunnicutt describes a female condom with a flange shaped shield retained in place by adhesive. The shield described, having a general triangular shape with a height of about 10-11 cm. and a maximum width of about 7 cm., cannot adhere efficiently to the skin surrounding the vulva because, as with the prior art, there is the presence of pubic hair in this region. In one embodiment, the shield is included in a rubber crotch section of a panty, but even here the regions lateral to the vulva are not sufficiently covered by this device to safely provide prophylactic or contraceptive function. Furthermore, no provisions are described to prevent seepage under the rubber crotch section. Additionally, no provision is addressed to use the device for anal intercourse.

U.S. Pat. No. 5,370,131 describes a thin-walled, exclusively male condom, which has one or more rings at the proximal end as a means to retain the tight-fitting condom anchored to the scrotum. A minimal size reservoir tip is inadequate to sufficiently capture and store seminal fluid thus allowing a distinct possibility of backflow and seepage.

U.S. Pat. No. 5,398,699 reveals a fairly loose-fitting, thin-walled, male condom, which is anchored at the base of the testicles. While reasonably functional and including a reservoir tip, the device is of such novelty appearance that many men may consider it emasculating and not take it seriously as an effective method for protection. It is design exclusively for the male user and it is not ergonomically structured for anatomical compatibility.

U.S. Pat. No. 5,433,219 reveals a thin-walled, female condom assembly designed exclusively as a liner for insertion into a passive sexual organ. Essentially this condom device is designed as an oversized conventional condom to function as a vaginal or anal liner and loosely fit the penis when inserted into the liner within the organ of the passive partner. A test tube shaped inserter tube is provided, however, it is not ergonomically designed to curve with natural anatomy. In addition, the rigid insertion tube requires an additional applicator, according to the inventor, in order to further facilitate full insertion. There is a minimal means for retaining the condom in the organ of the passive partner. The thin-walled, flexible sheath is structured with one or more rings to retain the device within the passive organ, however the device is too thin to retain its' shape, consequently it may be quite easily forced into the organ of a passive partner without his or her knowledge, thus compromising its primary purpose. The device, like many in the prior art, has no reservoir to help protect the passive partner from potential backflow of semen and consequential contamination.

U.S. Pat. No. 5,454,379 Describes a concept similar to U.S. Pat. No. 4,869,723. The device is primarily a male type condom with two sections; a thin membranous section and a second tubular section, which is slightly thicker but not stretchable. The device is manufactured by the traditional dipping method incorporating one or multiple dipping. The device is intended to be loose-fitting, applied to the penis and subsequently inserted into the vagina, however, because the tubular section is non-stretchable it requires, according to the inventor, that it “needs to be reasonably well fitted in size to the specific user's extended organ.” Considering this seemingly impractical possibility, this requirement would suggest the custom sizing for each individual user. Further, the device has no reservoir to help protect the passive partner from potential backflow of semen and consequential contamination. A means to retain the condom on the penis is provided with a conventional, tight fitting ring at the base of the penis and further complicated with an adhesive at the open end near the ring. This retaining feature presents one of the long standing complaints that many men find with the application of prior art; it is cumbersome and consequently discouraging, and can render such devices ineffective. Also, there appears to be no provision for a method of insertion or for an insertion device to pilot the condom into the passive partner.

Likewise, U.S. Pat. No. 5,318,042 and U.S. Pat. No. 5,718,236 have similar disadvantages of limited access and rigid construction relative to the scrotal sac. More apparent, unlike the present invention, these patents do not provide retaining mechanisms to prevent the condom from slipping partially or completely off during sexual intercourse.

U.S. Pat. No. 5,515,862 describes a thin-walled, female condom that requires adhesion to the walls of the vagina. Although identified as a female condom the sensory benefits provided are for the active male user, not the passive partner. The device is inserted into the vagina. A series of rings is provided to retain the device internally. Further, the device has no reservoir to help protect the passive partner from potential backflow of semen and consequential contamination. There is a collar provided, however it does not sufficiently protect the passive user from entry of the penis on the side of the collar's edge, unlike the present invention, comprising a cuff-shaped collar that prevents a side entry. Although the device is designed to resist, not prevent, passage of fluids between partners the device does not provide any significant improved feeling for either partner. The rings used to anchor the device can become an unnatural feeling obstacle for the active male user.

U.S. Pat. No. 5,803,085 reveals a non-elastic, unstretchable male type condom with two possible means to retain the condom at the base of the penis; a conventional elastic band/ring or a two ended tether which requires the active male user to physically tie the string mechanism manually. However impractical to fasten the tie can easily loosen during use, especially if lubricants are involved. Further, the device has no reservoir to help protect the passive partner from potential backflow of semen and consequential contamination. As previously noted with U.S. Pat. No. 5,454,379, because this device is non-elastic it would be difficult or impossible to estimate a universal, one-size-fits-all, length and girth.

U.S. Pat. No. 6,223,747 describes a thin-walled, expandable, female condom which eliminates the cumbersome conventional rolling application onto the penis. While functional both as a thin-walled vaginal liner forming barrier to fluids and also as a male condom the device is not provided any means of insertion into the vagina other than manually, or in the conventional manner when worn on the penis of the active male partner. Further, as with prior art, the device provides no reservoir to help protect the passive partner from potential backflow of semen and consequential contamination.

U.S. Pat. No. 6,341,607 reveals a thin-walled, female style condom that provides a vaginal liner for the female user. It does not have a means to retain the device in position inside the vagina. In addition a foam disk is place inside the device at the distal end. While this initially addresses the issue of backflow a flat foam disk can create bounce and fail to absorb semen completely and consequently jeopardize the efficacy of the feature.

German Patent Number 210,413 to Hollmann discloses a condom-like device having an outer ring. The outer ring of this invention radially extends the opening of the condom. This invention has no means for retaining the closed end of the device in the vagina. No provision is made to use the device for anal intercourse.

### OBJECTS AND ADVANTAGES OF THE PRESENT INVENTION

In view of the foregoing shortcomings, the disadvantages of the prior art, as well as other limitations not specifically mentioned herein, it should be apparent that there exists a need in the art for an improved, more reliable, more user-friendly and desirable condom. It is, therefore, a primary object of the present invention to resolve these issues and fulfill the need by providing a condom which will not only increase the safety standards of the device, create ease of use but will also simulate natural anatomical sensitivity for both partners.

There have been numerous proposals of condoms however the thick-walled condom, in accordance with the invention, results in an especially convenient, comfortable, desirable and efficacious condom, which has not been previously suggested.

Furthermore, the condom of the invention is suitable for large-scale production by conventional manufacturing processes, whereas the complexities of many of the proposals contained in the prior art would involve manufacturing difficulties making economic production on a commercial scale extremely difficult or impossible. Unlike prior art the condom of the invention is conveniently manufactured by casting, injection molding or thermal molding.

In the present art the collar covering the orifice 10 of the receptive partner causes the open end of the condom to perform as an effective barrier to pregnancy and/or the transmission of diseases since genital contact between the body fluids of the partners is prevented. If a penetration is made outside the condom device the fact that the collar 2 is present enables the partners to appreciate the problem immediately. This is a considerable advantage compared with known female condoms of prior art without such a shield.

By using a thick, soft, pliant, stretchable, elastomeric material the device of the invention can simultaneously stimulate the vaginal or rectal wall along with the penis 6, and specifically the glans 6a and shaft of the penis 6 can move in direct contact with the inside, natural-feel wall surface of the condom. The soft contact arrangement, whereby the inside diameter of the tubular protective device will easily stretch and conform loosely with gentle pressure to the overall size of the penis 6, provides more natural sensitivity for both partners.

An important feature of the unisex condom used as a receptive device of the present invention for a passive user is that it is a positive step that a passive partner, male or female, may take prior to intercourse to assure either contraception and/or prevention of disease transmission, without requiring the participation or acquiescence of the active male partner as with prior art. Due to the fact that a receptive condom may be inserted long before intercourse, it does not necessarily comprise any interruption or interference with sexual intercourse as with most prior art. The unisex condom of this invention provides excellent protection against the transmission of sexually transmitted diseases as well as efficient protection against unintentional pregnancies. The collar 2, because it covers the entry point of a receiving orifice 10 and reduces the risk of penetration outside the condom, makes the unisex condom device of the invention safer than any standard male condom. Unlike the standard male condom, it is unnecessary to remove the condom device of the invention immediately after ejaculation.

The soft wall thickness of the tubular device is intended to simulate the natural tissue of both the active and passive partners. Wall thickness can be varied depending upon the strength, stretch, and/or the desired softness and texture of the elastomeric material that is chosen for production of the invention.

Accordingly, besides the objects and advantages of the universal, unisex condom in my above patent, several objects and advantages of the present device are:

(a) to provide an improved, dual-function condom, which comprises a thick-walled, semi-rigid, soft and pliant material having a tubular shape and a predetermined length, which can be worn by either a male covering the penis or otherwise inserted into the orifice of a passive partner, such as a vagina or anus, for superior protection against sexually transmitted disease (STD), sexually transmitted infection (STI) and/or for the prevention of pregnancy.

(b) to provide a more desirable and more comfortable protective safety condom device which gives a comfortable fit, increased sensitivity that simulates natural anatomical feeling, tactile sensation, enhanced and maximized male (organ) stamina and enhanced sexual enjoyment for both partners.

(c) to provide a desirable condom that resolves many important issues regarding function and structure such as; leaks, tears, breaks, discomfort, slippage, unintended removal and difficult application, many of which have prevented the conventional condom from being used as effectively and as extensively as needed in the HIV/AIDS crisis.

(d) to provide a thick-walled condom device of such durable construction that it may optionally be reused after proper sterilization.

(e) to provide a convenient condom device, which is easy, and instant to apply onto a penis or to easily insert into an orifice such as a vagina or anus of a passive user and optionally, using a stretchable strap providing means for retaining the condom with the active male partner, it is unlikely to unintentionally be removed.

(f) to provide an enhanced condom with a retaining strap, which will apply pressure behind the base of the scrotum for an active male partner to trap blood flow away from the user's erect penis and consequently assist to maintain the active male's erection during intercourse.

(g) to provide a solo operated condom device, which can be used by a male for self stimulation and also for the medical purpose of collecting sperm from a sperm donor for use by a fertility clinic.

(h) to provide a protective condom device that is simple and inexpensive to fabricate and mass produce with existing manufacturing methods.

(i) to provide a thick-walled protective condom of stretchable elastic material, which when in place, covers and conforms to the overall shape of an erect penis of any length and girth.

(j) to provide a superior condom which increases responsibility for birth control and safer sex for both male and female partners.

(k) to provide a superior condom which is economical, especially in light of the optional re-use after proper sterilization.

(l) to provide a condom for females that does not require installation by a physician.

(m) to provide a condom with a superior reservoir that will contain and trap semen including a reservoir with an absorbent foam disk insert.

With the foregoing and other objectives, advantages and features of the invention that will become hereinafter apparent, the nature of the invention may be more clearly understood by reference to the detailed description of the invention and the illustrated views in the drawings contained herein.

### BRIEF SUMMARY OF THE INVENTION

The invention is based, in part, on the awareness that a desired protection against 1. the transfer of infectious matter, STD, STI, including the AIDS virus and 2. unwanted pregnancy is obtained when a condom-like or tubular protective device, is used during sexual intercourse.

Briefly described, these and other objects are accomplished according to the present invention by providing a receptive, protective safety condom device adapted to be worn by a sexually passive partner, male or female, comprising a thick tubular sheath. Used as a receptive condom the device may include an optional applicator to assist with insertion into a passive orifice. The device is sized to line and fill the sexual organ of a passive partner, receive a penis at the open end, yield to a penis to stretch within the passive sexual organ and enclose and loosely grip the penis.

Within the context of this invention, a passive sexual organ is any organ that receives an erect penis during sexual relations (e.g., intercourse). The condom device is placed over the male genitalia prior to sexual contact or inserted into a vaginal or anal orifice of a passive, receptive partner.

This dual function, universal, unisex condom is designed to overcome the objections to use of prior art male and/or female condoms and to prevent the transmission of STD or STI by precluding skin-to-skin genital contact during intercourse.

The collar at the proximal open end 2a functions as a protective shield 2 which folds over itself and extends in a direction perpendicular to the said tubular sheath FIG. 1 so as to prevent pushing the device fully into a passive partner's orifice 13, 14 during sexual activity.

Optionally, the collar at the proximal open end may also include an integral elastic strap of the same contiguous construction and material as the tubular sheath and may be stretched behind the scrotum of the active male partner to secure it in place and prevent unintentional or accidental removal. Further, the elastic strap may serve to increase or support male stamina and help to maintain an erection for an active male user.

The closed end covering the glans penis may optionally include a smaller diameter reservoir section inside the distal end of the device as a receptacle for ejaculate. The reservoir may be fitted with a sponge-like disk to absorb semen and help to further prevent unwanted backflow.

Venereal disease prophylaxis and contraception is achieved by providing a condom device as described above, and positioning it over the male genitalia prior to sexual contact or by insertion into the orifice of a sexually passive partner.

A condom in accordance with the present invention herein described increases its' effectiveness as an impervious barrier to the spread of disease. Moreover, such a condom is not prohibitively expensive to manufacture by casting, injection molding or thermal molding.

It may be appreciated that there are various features described in the many embodiments of the present invention. It is within the scope of the invention to combine these features in various configurations as required, without departing from the intent or overall purpose of the invention.

### BRIEF DESCRIPTION OF THE DRAWINGS—FIGURES

FIG. 1 shows a profile view of a first preferred embodiment of the present invention.

FIG. 2 shows a perspective view of the preferred embodiment of the present invention wherein a tubular protective device has an elongated, elliptical, tubular body with a collar 2 portion. This embodiment includes an strap 3 at the proximal open end as retaining means.

FIG. 2a shows a profile side view of a first preferred embodiment of the present invention.

FIG. 3 shows a cross-sectional view of the preferred embodiment including a reservoir.

FIG. 4 shows a profile and a partial cross-sectional view of the preferred embodiment of the present invention placed upon the penis 6 with a retaining strap 3.

FIG. 5 shows a cross-sectional view of the female reproductive organs with the condom in place in the vagina 17 of a female user.

FIG. 6 shows a perspective view of the preferred embodiment of the collar 2 in place covering the labia majora 10 of a passive partner.

FIG. 7 shows a perspective view of the preferred embodiment prior to complete placement of the condom onto a penis and the application of the retaining strap 3.

FIG. 8 shows a condom according to this invention in assembly with an insertion applicator wherewith the condom body may be fully inserted into the sexual orifice of a passive partner by positioning of the insertion applicator rod into the condom and then inserted together into the vagina or anus of a passive user.

The invention is described in more detail with reference to the figures, which show desirable embodiments of the tubular protective device according to the invention.

### REFERENCE NUMERALS

1 Closed distal end
2 Collar
2a Open proximal end
3 Retaining strap
4 Neck
5 Reservoir
6 Penis shaft
6a Glans penis
7 Scrotum
8 Receiving condom
9 Vulva
10 Receiving sexual orifice
11 Urinary bladder
12 Cervix
13 Vagina
14 Anus
15 Rectum—rectal wall
16 Applicator
17 Labia majora
18 Foam disk insert

### DETAILED DESCRIPTION OF THE INVENTION

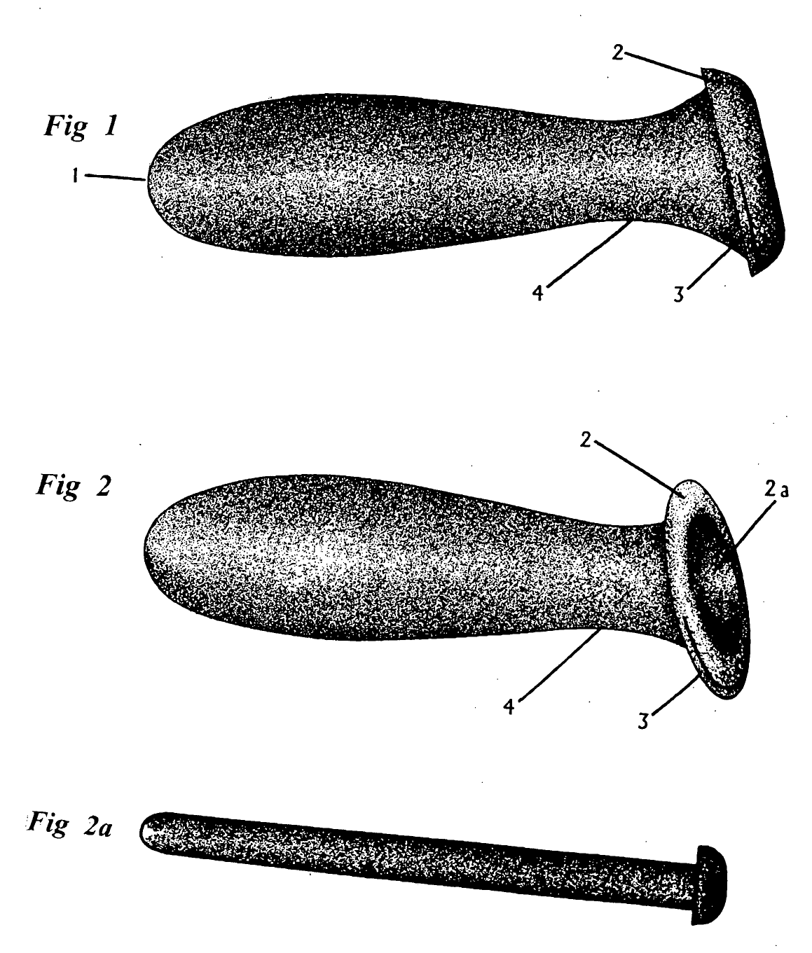

FIG. 1—Preferred Embodiment

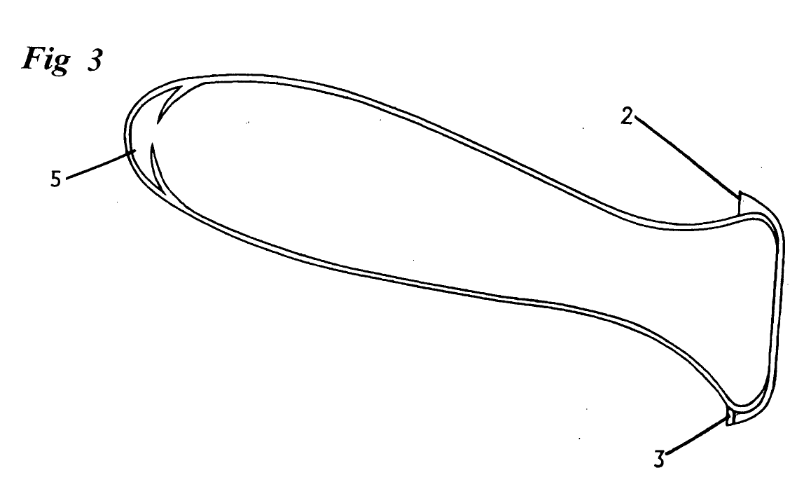

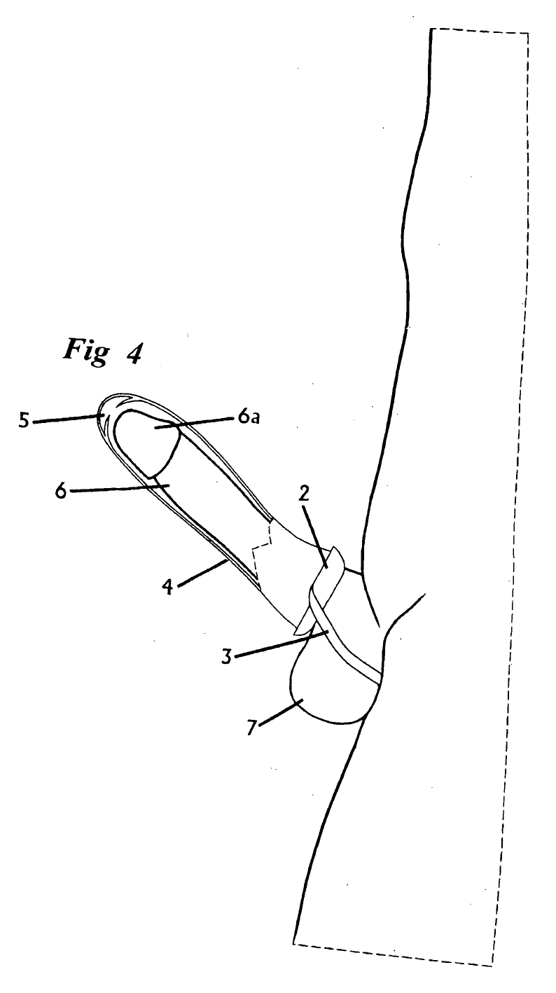

In a first preferred embodiment the condom device of the invention includes an elliptical, elongated tube formed of a thick, pliant, polymeric material impervious to spermatozoa and pathogenic organisms. It is structured as a heavy-gauge, thick-walled condom device FIG. 3 of a tubular construction that includes a single continuous, elastic tubular sheath FIG. 1 with a proximal open end 2 and a distal closed end 1. When in place, the condom sheath conforms to the general shape of the covered male genitalia FIG. 4 and the closed end 1 of the device covers the glans 6a penis. The shaft 4 of the tubular device covers the entire penis 6, including the base of the penis. The device may include a strap 3, which when stretched, fits around and behind the entire scrotum 7 of the active male. The preferred embodiment is constructed of thick, heavy gauge, super stretchable, non-burstable, semi-rigid material, said material being selected from a group consisting of polymers, USP class six thermoplastic elastomers (TPE), latex, silicone rubber or other semi-rigid, soft, pliant, stretchable, biocompatible material or combinations and derivatives thereof.

FIGS. 3, 4, 5, 7 and 8—Additional Embodiments

Additional embodiments are shown in FIGS. 3, 4, 5, 7 and 8. FIG. 3 illustrates the condom will fit within the internal anatomical structure of the vaginal 13 or rectal 15 cavity. The invention further includes at the open end 2a an optional collar 2 which functions as a shield or barricade to facilitate and prevent the device from being pushed completely into a receptive orifice 8, 14 of a passive partner.

FIG. 3 shows a cross-sectional view of the first embodiment. The material of the shaft wall preferably comprises a thickness ranging from 1/64 inch to ⅛ inch, however the preferred dimensions of the tubular device can vary to those that provide the best fit for proper use, maximum sensitivity and safety for both partners.

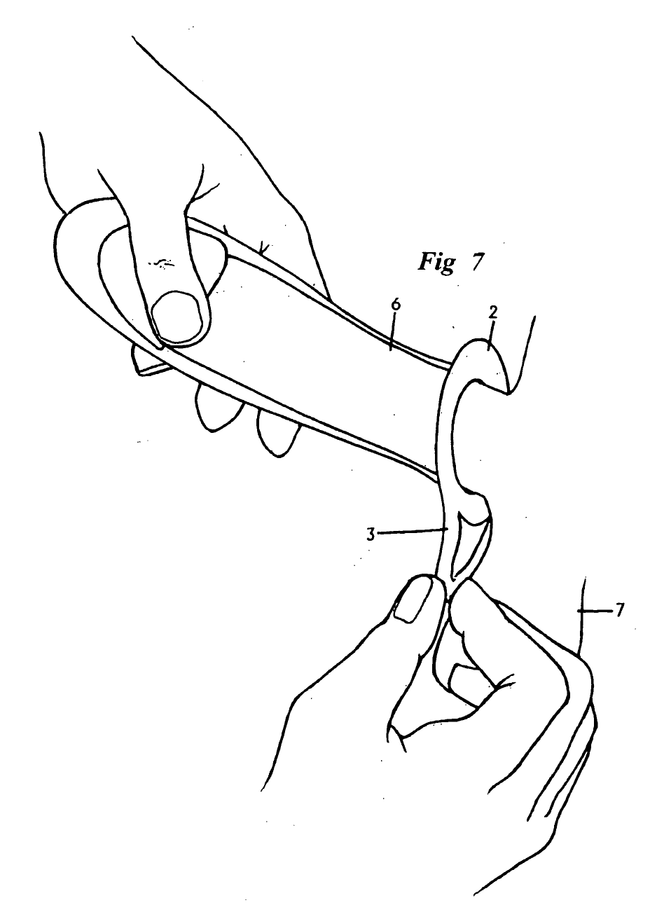

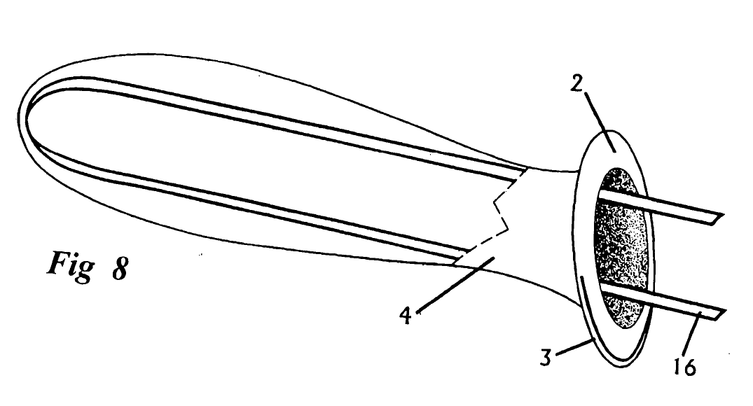

FIG. 7 shows a partial cross-sectional view of the device as described in the first embodiment, in a condition of completed placement over the male genitalia. A smaller diameter, closed collection well 5 optionally includes a receptacle for ejaculate from an active, penetrating male partner and formed inside the tubular condom device at the closed distal end 1 and may also include an absorbant, sponge-like, foam disk within to further facilitate collection and prevention of back flow following ejaculation by an active male partner.

An additional embodiment is a condom as described in the first embodiment and may include a plurality of protuberances, which may be formed along the shaft of the condom in a wide variety of shapes, ridges textures and configurations including, but not limited to accordion and/or pleated designs to provide added stimulation during coitus for the partners. Also, the device may include, but is not limited to, accordion and pleated flexible tubes. The device may be longitudinally stacked in accordion style for purpose of compact packaging. These may be configured as required for a desired number, orientation or combination for maximum benefit and results. Such components may be included or omitted.

A further embodiment of the invention when used as a receptive or female condom, shown in FIG. 8, shows a partial cross-sectional view of the condom of FIG. 2 in assembly with a long applicator 16 used to insert the condom into the vagina 13 or anus 14. The collar 2 is larger in diameter than both the medial tube portion 2 and larger than the nominal vaginal 13 or anal 14 opening in FIG. 6.

FIG. 4, is shows the tubular portion and the flared collar portion 2 and retaining strap 3 described previously. The integrated strap 3 acts to anchor the condom onto the penis and to increase pressurized stimulation behind the scrotum 7 of the active male partner, which promotes sustained erectile stamina.

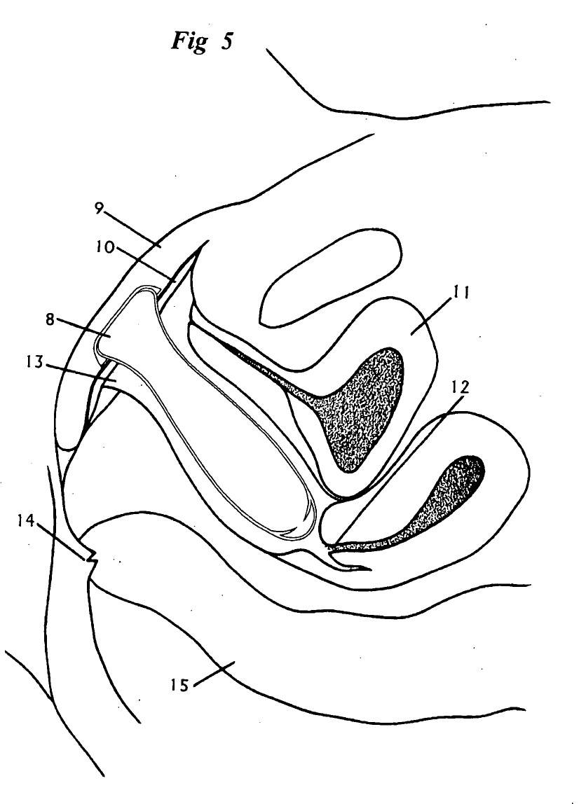

The protective device of the present invention is a condom designed for use by females FIG. 5 or by males FIG. 4, which includes an elliptical, elongated tube formed of a pliant, heavy gauge, elastomeric material impervious to spermatozoa and pathogenic organisms, and including a closed distal end 1. The proximal end 2a is open and may optionally be provided with a cut-out or opening 3 formed at the open end 2 to provide a semi-circular, strap means of retaining 3 the tubular protective device on the erect penis 6 of a male partner.

The condom of the invention designed for use by females or by males that includes a thick, elongated, tubular sheath formed of a pliant, heavy gauge, elastomer material impervious to spermatozoa and pathogenic organisms, and including an open proximal end and a closed distal end. The proximal end is optionally provided with a semi-circular cut section or opening formed at the edge of the collar to provide a strap-styled means of retaining the tubular protective device on the erect penis of an active male user.

Further, the art of the present invention provides an applicator 16 for the purpose of inserting the condom inside a receptive orifice of a passive sexual partner.

Pursuant to another feature of the present invention, the device may comprise a lubricating substance, with or without Nonoxyl 9, inside and/or outside the device. The lubricant may include a spermicidal agent, a bactericidal agent, and/or an antiviral agent. The lubricant is, desirably, applied to the tubular protective device prior to or in connection with the insertion of the tubular protective device. The lubricant is applied to the inner side of the device in order to reduce friction during contact with the penis. If desired, a lubricant may also be applied to the exterior side of the tubular sheath to reduce friction during contact with the wall of the vaginal or rectal organ. Application of a lubricant to the exterior side of the tubular device can facilitate smooth, comfortable, uninterrupted insertion of the device into the passive orifice at the time of or prior to intercourse.

The prophylactic device of the invention may comprise an optional series of projections ribs, bumps, ridges, pleats, accordion folds and/or various textures and combinations thereof for enhancing genital stimulation during intercourse for either or both partners.

Pursuant to a further feature of the present invention, the condom is provided with a semi-circular retaining strap in a region about the outer edge of the collar shield at the open end with an opening for permitting a means of retaining the device to the scrotal sac and testicles of an active male partner.

The condom of the present invention increases its' effectiveness as an impervious barrier to the spread of disease. Moreover, such a condom is not prohibitively expensive to manufacture by casting, injection molding or thermal molding.

The outer cuff-shaped, cushioning, collar fits onto and covers over the outside of the sexual orifice of a passive user. The purpose of the cuff is to provide coverage and a barricade with a certain degree of cushioned rigidity, so that the tubular condom cannot get pushed or pulled inside the passive sexual organ during vigorous sexual activity. The condom of the invention may optionally be sized for added comfort, for example within a range of at least four sizes; small, medium, large, and extra large. The size needed can be determined by the size of the passive sexual organ or the length and girth of the erect penis of the active male partner, however the device is intended to provide maximum stretch capabilities that would create a one-size-fits-all condom and therefore not require an assortment of size options for the consumer.

Although only preferred embodiments are specifically illustrated and described herein, it will be appreciated that many modifications and variations of the present invention are possible in light of the teachings contained herein and without departing from the scope and intended spirit of the invention. For a more complete understanding of the invention by those skilled in the art, the following examples are given by way of illustration. Relative to various aspects thereof the present invention is shown in the drawings in which like items are identified by the same reference numerals but different alphabetical suffixes.

The present invention generally comprises a condom for use in preventing both contraception and transmission of venereal diseases. A salient feature of the invention is that it can be used by either male or female partners prior to intercourse, rather than exclusively by an active male, as is known in the prior art.

The thickness of the wall FIG. 3 of the tubular device must be such that the resistance to mechanical stresses of the condom laid down by standards in force in different countries is guaranteed. The condom device, is preferably of such durable construction that it may optionally be reused after proper sterilization. The dimensions of the condom of this invention are such that they allow movement of any size penis, small or large, relative to the condom during sexual intercourse.

The protective device of the present invention is a condom designed for use by females FIG. 5 or by males FIG. 4 which includes an elliptical, elongated tube formed of a pliant, heavy gauge, elastomeric material impervious to spermatozoa and pathogenic organisms, and including a closed distal end 1. The proximal end 2a is open and may optionally be provided with a cut-out or opening 3 formed at the open end 2 to provide a semi-circular, strap means of retaining 3 the tubular protective device on the erect penis 6 of a male partner.

The invention in the present art is made of a flexible, thick-walled condom, elongated and tubular in shape and has an open end and a closed end. The condom is preferably made of a polymeric, elastomeric materials comprising various formulations and derivatives and being selected from a group consisting of polymers,

USP class six thermoplastic elastomers (TPE), silicone rubber, latex or other semi-rigid, soft, pliant, stretchable, biocompatible material and combinations or derivatives thereof, which has a semi-rigid yet soft, gelatinous texture, and feel. Optionally conventional latex, can be used to manufacture the heavy gauge, thick-walled tube of this invention.

A further aspect of the present invention, shown in FIG. 4 is directed toward a tapered neck 4 portion and enlarged distal closed end of the shaft of the tubular device which fits closely to the penis to prevent the condom from being pulled out of the receiving orifice of the passive partner during intercourse. The generally cylindrical tubular neck 4 of the condom, as described previously, is provided with one or more contours and optionally an additional ridge, texture or plurality of same circumferentially therealong.

The device of the invention can be manufactured by casting, injection molding or thermal molding methods that are not presently used within the condom industry for fabricating either male or female condoms. Further there are no condoms presently manufactured using these methods. The specific method chosen to manufacture the device of this invention varies with the particular formulation chosen for the selected material. Heated or liquefied elastomeric material is poured or injected into a mold, then cooled and removed from the mold.

Although not limited to any specific margins, a preferred embodiment a wall thickness in FIG. 3 for the device is between 1/64″ and ⅛″. A wall thickness within these dimensions will provide a tensile strength far beyond the industry standards for traditional latex condoms and thus exceed compliance with the standards of the American Society for Testing and Materials and FDA requirements applicable to condoms. The internal or inner diameter of the tubular protective device in its stretched state is desirably of a sufficiently large dimension to permit internal movement of a penis 6 with respect to the protective device during sexual intercourse.

The closed distal end covering the glans 6a penis includes a smaller diameter reservoir 5 inside the closed end of the device as a receptacle for ejaculate. This reservoir may be further improved by including an absorbent, sponge-like material to absorb fluids and help to prevent backflow which is common in prior art.

The internal or inner diameter of the tubular protective device in its unstretched state is desirably of a sufficiently large dimension to permit penetration and movement of a penis 6 with respect to the protective device during sexual intercourse.

The wall thickness of the tubular protective device can vary greatly. Preferably a thicker wall for the device allows more sensitivity during coitus. Wall thickness can be varied depending upon the strength of the elastomeric material that is chosen for the device.

Advantages

The unisex condom FIG. 5 capable of being inserted into the vagina 13 or anus 14 of a passive user comprises a flexible, thick-walled tube FIG. 3 having a closed end 1 and an open end 2a. The open end 2a is provided with a strap 3 which has approximately ⅓ the size of the circumference of the edge of the collar at the open end 2a and which delimits the shield from entering the passive orifice with respect to the tube.

The outwardly extended portion of the tubular protective device provides a collar 2 that functions as a barricade having a larger diameter than the shaft of the tubular sheath to prevent the open end of the tubular protective device from being pushed into the vaginal 13 or anal 14 orifice of a passive partner, thus preventing the exchange and contamination of secretions and body fluids between partners during sexual intercourse.

The collar 2 at the proximal end 2a which folds over itself and extends in a direction perpendicular to the tube FIG. 1 so as to prevent pushing the device fully into a passive partner's orifice 13, 14 during sexual activity. Other characteristics of the invention are further described, in a non-limiting way, in the examples included in the descriptions and illustrations herein.

The tubular protective device may be further prevented from unintentional removal by optionally utilizing a strap 3 feature by the active male partner. Preventing unintentional removal of the device from the penis during intercourse is accomplished by a means of a retainer or retaining means. In the present art the means for retaining is formed with a semi-circular slit or opening 3 located along the outer edge of the angularly positioned, proximal, open end 2a.

In use, the condom, in accordance with the invention, can be held in its longitudinally, flat extended state FIG. 7 and the closed end of the condom can be pushed into the vagina 13 or the rectum 14 by the penis as it penetrates at the beginning of sexual intercourse with a collar of a diameter ensuring that the open end 2a remains outside 8 during initial insertion and throughout the coital act. A flexible plastic applicator 16 is optionally provided as means to insert the condom device inside a sexual orifice of a passive partner.

Additionally the strap 3 is stretched and placed around and behind the scrotum 7. Following correct placement this strap serves two purposes for an active male partner; 1. it will anchor the device to the erect penis to prevent unintentional removal or slippage and 2. it will apply pressure behind the base of the scrotum, which serves to trap blood flow away from the penis and consequently assist to maintain the active male's erection during intercourse.

The collar is of a predetermined dimension that completely covers the orifice 10 of the passive partner. Such protection is magnified in the device of the invention in FIG. 2, which desirably has a large inside diameter FIG. 4 to allow movement of a penis 6 within the walls of the tubular device FIG. 4. The walls of the tubular device can be held in a relatively immovable condition within and against the vaginal or rectal wall by the curved, tapered neck 4 approximately one third of the way along the shaft near the open end of the tubular device, which serves as a means for retaining the device internally 8 with muscle contraction by the passive partner.

The present invention can have a large inner diameter that causes the tubular protective device to also function as a loosely form-fitting condom for an active male partner FIG. 4 used in the manner of a conventional latex condom, when in use with the strap.

The condom may be prevented from unintentional removal by optionally utilizing the strap 3 feature by the active male partner. Preventing unintentional removal of the device from the penis during intercourse is accomplished by a means of the retainer strap or retaining means. In the present art the means for retaining is formed with a semi-circular slit or opening 3 located along the outer edge of the circular shield 2 at the open, proximal end 2a. This feature essentially creates the strap 3, which is stretched and placed around and behind the scrotum 7. Following correct placement the strap serves two purposes for an active male partner; 1. it will anchor the device to the erect penis to prevent unintentional removal or slippage and 2. it will apply pressure behind the base of the scrotum, which serves to trap blood flow away from the penis and consequently assists to maintain the active male's erection during intercourse.

Optionally, a generally form-retaining applicator 16 may be provided within the condom for purpose of insertion of the condom into the orifice of the passive partner 8, 14. The applicator 16 in this embodiment comprises a smooth, flexible solid probe or tubular rod of polymer or plastic material having sufficient rigidity to guide, pilot and insert the condom device into the vaginal or anal orifice. The interior of the condom is provided with a lubricant to release the rigid applicator and permit its withdrawal therefrom after insertion of the condom. In this embodiment the condom forms a closed lining of the vagina 13 or rectum 14, with the proximal end of the condom extending outside of a passive orifice, to prevent epidermal-genital contact during intercourse and to prevent the release of semen within the orifice.

Insertion into the vaginal 13 or anal 14 orifice of the condom device of the invention can also be self-inserted by either an active or a passive partner by manually pushing the condom inside a sexual orifice. The condom can also be inserted by means of the insertion applicator prior to intercourse. The plastic insertion applicator 16 is provided to facilitate easy insertion. The device can also be inserted into the orifice of a passive partner in the traditional manner wherein an active male partner places the device over the penis before penetration is made. The erect penis of the active male is axially movable relative to the condom. During the insertion into the vagina 13 or the anus 14, the curved, tapered neck section 4 of the shaft of the tubular wall is further held in place by muscle contraction in orifice of the passive partner; vaginal, anal or oral.

With regard to FIG. 3, the basic configuration the present invention includes a condom formed of a generally cylindrical tube 1. The tube includes a distal end 1 having an integrally formed closure of generally hemispherical or smoothly rounded configuration, and a proximal end 2a that is open.

During insertion of the condom through the relatively narrow opening of a passive orifice the tubular condom resiliently compress radially inward toward the axis of the tubular portion thereby diminishing in diameter for purposes of insertion.

It may be appreciated that during insertion of the condom by the applicator 16 or the like, the tensile force applied axially to the condom by the applicator causes the condom to elongate elastically and constrict diametrically. When the applicator is withdrawn, the elastic memory causes the tapered neck 4 portion to resume its original form with the constricted diameter returning to normal and expanding against the wall of the orifice of a passive partner to retain the condom internally.

After complete insertion of the tubular device 8 into the vagina 13 or anal 14 orifices, the tubular condom resiliently expands and resumes a larger diameter. The increase in diameter along with the narrow tapered neck of the shaft retains the condom in place in the sexual orifice of a passive user, during and after intercourse.

In a preferred advantageous embodiment, the contoured, tapered neck 4 section of the shaft of the condom device permits the passive receptive partner to use internal muscles to grasp and consequently help to hold the device inside the orifice during sexual intercourse. However, the condom can be removed by manually grasping the shield-like collar 2 and pulling the device from orifice 13, 14 of a passive partner, with the tubular shaft collapsing during withdrawal.

The diameter of the condom is being sufficiently small or narrow to facilitate insertion or removal of the condom. After initiation of intercourse, if the diameter of the penis 6 is greater than diameter of the condom shaft the pliant elastomer material will expand sufficiently to accommodate any size of the penis 6 and assure that the condom will not engage or otherwise be retained on the penis at the time of its removal. Thus the condom will remain in the receiving orifice of the passive partner to only be removed manually after intercourse.

After initiation of intercourse, the proximal open end 2a of the tubular constriction expands elastically to admit the penis 6 and thereafter contracts about the overall shape of the penis and is therein retained using the retaining strap. The condom 4 is thereafter secured to the penis, and moves in concert therewith. Seminal fluid released within the condom is retained therein or collected in the optional reservoir 5 therein after the completion of intercourse.

Furthermore, after initiation of intercourse the insertion of the penis within the interior sidewall 4 of the condom causes a “plug” effect at the tapered neck section of the tubular device that further retains the unisex condom of the invention and prevents its accidental removal during intercourse.

A lubricant is, preferably, applied to the tubular protective device prior to or in connection with the insertion process into the sexual orifice of a passive partner. A lubricant is applied to the inner side of the device in order to reduce friction during contact with the penis. If desired, a lubricant can also be applied to the exterior side of the tubular protective device to reduce friction during contact with the organ wall of the vagina 13 or rectum 14. Application of a lubricant to the exterior side of the tubular protective condom device can facilitate smooth, uninterrupted insertion of the device into the passive orifice at the time of intercourse. Further, a lubricant, with or without Nonoxyl 9, may be pre-applied by the manufacturer to the inside and/or outside of the tubular protective device.

### CONCLUSION, RAMIFICATIONS AND SCOPE

The present invention relates to condoms and it is especially concerned with condom devices which function as a receptive condoms 8 that line the wall of the vagina 8 or rectum and which also function as a male condom which may be initially donned by a male FIG. 4 and dimensioned to loosely cling with light pressure closely around the penis 6 and enclose the penis 6 at least over the length of the condom received within the sexual orifice of a passive partner, such as the vagina 13 or the anus 14. For convenience such a condom is referred to herein as a “unisex condom”.

Traditional as well as newly designed, thin, form-fitting condom devices do not permit adequate sensitivity for a male during sexual intercourse because a form fitting condom moves along with the penis 6 and prevents direct tactile contact and friction between the vaginal or rectal wall of a passive partner and the penis 6 of an active male partner during sexual intercourse.

The undesired effect of a traditional, thin, form-fitting, rolled condom discourages their use by many members of the population. In addition, a tubular protective device commonly referred to as a ‘female condom’ having a large inner diameter merely functions as a thin liner for the vaginal wall or “vaginal pouch”.

The present art changes the basic approach of traditional, thin-walled, rolled condom design. The following advantages and benefits of the present invention are illustrated in the drawings included herein.

The present invention is intended for protection against transfer of infectious matter during sexual intercourse, for use as a contraceptive and also as a stimulating device for male and/or female partners during sexual intercourse. Additionally the device of the invention may be used by a male as a self-operated stimulating device and also for the collection of sperm from a sperm donor as required by a fertility clinic. Furthermore, the universal, unisex condom of the invention has additional advantages in that;
it offers for the first time a comfortable, natural fit, ergonomically designed, unisex condom device that provides increased sensitivity simulating natural tactile sensation.
It is manufactured as a non-rolled, flat packaged device, which provides easy, instant application, by the user who can be either female or male.
it enhances male (organ) stamina and sexual enjoyment for both the active and the passive sexual partners.
it will not break, leak, slide off the penis or slip out from a sexual orifice due vigorous friction during sexual intercourse under normal use. Consequently it provides ultimate protection against STD, STI and unwanted pregnancy.
it incorporates a contoured, tapered neck 4 portion between the collar 2 at the open proximal end 2a and the closed distal end 1 of the tube. The tapered neck serves as retaining means for the passive partner's use. This reduces still further the risk of unintentional removal or slippage of the condom. The condom device 8 may be held in position by muscle contractions inside the sexual organ of a passive partner during coitus.
the reservoir is of the type comprising a foam disk insert 18, which readily absorbs semen helping to prevent backflow and possible contamination of the passive partner.

The present invention has been considered in recognition of the enormous increase in sexually transmitted diseases such as AIDS and a resurgent interest toward the development of reliable, convenient contraceptive/prophylactic devices to be worn by a passive sexual partner during sexual activity.

In accordance with the present art, a method has now been determined for improving the user protection, safety and desirability associated with condoms by use of injection molding methods as a preferred manufacturing process. This new approach to the design, structure and mechanics of a condom provides a thick barrier rather than for a thin barrier produced by the dipping method used for conventional male condoms.

Standards for condoms within the industry, typically, do not define the inner diameter of a condom, but define the acceptable width of the condom when it is laid flat on a surface. A condom having a width of about 48 millimeters to about 52 millimeters is considered, within the industry, to be form fitting. Contoured or loose fitting condoms have a width of about 50 millimeters to about 53 millimeters. For the present invention predetermined dimensions will vary according to the stretch produced and by the specific formulation of the elastomeric material that is used.

Of course, the thickness of the wall must be such that the resistance to mechanical stresses of the condom laid down by standards in force in different countries is met or exceeded. The dimensions of the condom device are such that they allow movement of the penis 6 relative to the condom during sexual intercourse, as well as optionally by the partners that will allow the penis 6 along with the condom to move together in concert inside the orifice a passive partner

The wall thickness, regardless of the material from which the device is manufactured, must provide a tensile strength to comply with the standards of the American Society for Testing and Materials and also meet or exceed FDA requirements. In a preferred embodiment a wall thickness FIG. 3 is between 1/64 and 1/8 inch is desired, however it can vary with less or more thickness and strength and still exceed various international standards.

Further the protective device may optionally include one or more devices or constructions for retaining the closed end of the device in a vagina 13 or rectum 14 of a user and/or prevent the device from being forced completely inside 8.

Although the invention has been described in terms of particular embodiments and applications, one of ordinary skill in the art, in light of this teaching, can generate additional embodiments and modifications without departing from the intended spirit of or exceeding the scope of the invention. Thus the scope of the invention should be determined by the appended claims and their legal equivalents, rather than by the example given.

What I claim as my invention is:

1. A method of manufacturing a thick-walled, tubular condom, comprising the steps of
first, casting a mold for the thick-walled, tubular condom,
second, inserting a material into the mold, where the material is selected from a group consisting of polymers, thermoplastic elastomers, silicone rubber, latex or other comparable, semi-rigid, soft, pliant, stretchable and/or biocompatible materials, or derivatives and combinations thereof,
third, allowing the molded material to cure,
fourth, removing the condom from the mold,
fifth, forming the condom into a substantially flat shape, wherein the condom readily folds in a bellows manner for the condom to collapse longitudinally,
sixth, applying packaging means to the condom such that the condom is packaged for sale,
where, the condom to be produced through the method described in this claim comprises a thick-walled, universal, unisex, safety device for protecting against communicable diseases transmitted by the exchange of bodily fluids and for preventing unwanted pregnancy, comprising: a closed distal end, an open, proximal end, and a cylindrical wall section, where the condom is generally an elastic, thick-walled, shape-retaining, tubular sheath of longitudinal axis with an inside and outside surface, comprising a semi-rigid, soft, pliant, stretchable wall material defining an open proximal end and a closed distal end, having a generally phallic shape, wherein the shape-retaining, tubular sheath is adapted to receive a male sex organ via the open, proximal end, and additionally being adapted to be inserted into a sexual orifice of a passive partner such as a vagina and an anus, and wherein, where the condom is constructed from an elastomeric material comprising a super stretchable, non-burstable, semi-rigid material selected from a group consisting of polymers, USP class six thermoplastic elastomers (TPE), silicone rubber, latex or other semi-rigid, soft, pliant, stretchable, biocompatible material or combinations and derivatives thereof, whereby said shape-retaining, tubular sheath is adapted to provide soft, natural tactile sensation, and, wherein said elastomeric material surrounds the penis completely enclosed, said shape-retaining tubular sheath having a sufficiently large diameter, wherein movement of the penis is made possible within the condom during coitus, whereby fluid communication is prevented between the interior and the exterior thereof, and, where, the condom does not have to be rolled out over the male sex organ to function.

2. The method of claim 1, where the forming of the condom into a substantially flat shape is performed by laying the condom flat.

3. The method of claim 1, where the forming of the condom into a substantially flat shape is performed by collapsing the condom longitudinally wherein the condom readily folds in a bellows manner for the condom to collapse longitudinally.

4. The method of claim 1, where the mold has a predetermined size and shape adapted to form a condom of a specific length and diameter.

5. The method of claim 1, where the method of inserting a material into the mold is injection molding.

6. The method of claim 1, where the method of inserting a material into the mold is thermal molding.

7. The method of claim 1, where the condom to be produced through the method described in this claim comprises a cylindrical wall section of the shape-retaining condom is thick enough to allow a passive sexual partner to insert the condom into a vagina or anus without assistance from an active sexual partner.

8. The method of claim 1, where the condom to be produced through the method described in this claim comprises a collar extending outwardly as a contiguous shield circumferentially formed around the longitudinal axis of the tubular sheath of the condom, where the collar comprises a tapered neck at the open, proximal end and an internal diameter adapted to receive the male sexual organ and, where the collar comprises a barricade to cover the vulva or anus during coitus, such that the condom is prevented from being forced into said orifice of a passive sexual participant during sexual intercourse, and where the collar provides for female clitoral stimulation.

9. The method of claim 8, where the collar of the condom to be produced through the method described does not contain any rings or other metal or hard plastic devices to keep the collar at the surface of the entrance to a vagina or anus.

10. The method of claim 8, where the collar of the condom to be produced through the method described is larger at the open proximal end than the diameter of the cylindrical wall section of the condom, such that the collar is retained outside the vagina or anus during sexual intercourse, and where the tubular sheath portion is of a size to form a loose fit around the penis, wherein the wall of the tubular portion is formed with said flexible, semi-rigid material, and whereby the wall facilitates insertion into the sexual orifice of the passive user.

11. The method of claim 1, where the condom to be produced through the method described in this claim additionally comprises a generally tubular shape-retaining body, and has a first means for retaining and securing to an active male wearer during intercourse, and a second means for retaining and securing within the orifice of a passive wearer during intercourse.

12. The method of claim 1, where the condom to be produced through the method described in this claim additionally comprises a second means for retaining and securing within the orifice of a passive wearer during intercourse is a collar, where the collar extends outwardly as a contiguous, shield circumferentially formed around the longitudinal axis of the shape-retaining, tubular sheath of the condom.

13. The method of claim 12, where the condom to be produced through the method described in this claim is constructed such that the first means for retaining and securing the condom to the penis of an active male wearer during intercourse is a semi-circular, flexible, elastic, radially extending retaining strap which is circumferentially and contiguously formed adjacent to and in communication with the outer edge of the collar at the proximal, open end, said retaining strap adapted to be stretched away from the collar to be arranged around the scrotum and testicles of the active male wearer and retained there snuggly, thereby providing a securing mechanism in communication with the shape-retaining, tubular sheath for securing the device to the active male wearer, and, where the retaining strap comprises a stretchable material coupled to the collar at the proximal end thereof, and, where the material is selected from the group consisting of biocompatible polymers, thermoplastic elastomers, silicone rubber latex or comparable combinations and derivatives thereof.

14. The method of claim 12, where the condom to be produced through the method described in this claim is constructed such that the second means for retaining and securing comprises a shape-retaining, tubular shaft with a graduated diameter whereby the shape-retaining, tubular sheath is tapered and contoured to more readily be retained with muscle contraction by a passive user within a sexual orifice.

15. The method of claim 14, where the condom to be produced through the method described in this claim is constructed such said shape-retaining, tubular sheath comprises the closed, distal end, the tubular sheath portion shaped such that, when the condom is in place over the male genitalia, the shape-retaining, sheath covers the penis of any length and girth, including the base of the penis shaft, the strap is harnessed snuggly around the scrotum and retained by the posterior of the scrotum, wherein the condom is comprising a penis covering segment, the collar, the seminal reservoir or reservoirs, the graduated diameter means for retaining within the sexual orifice of the passive user, an insertion applicator and the strap means for retaining.

16. The method of claim 12, where the condom to be produced through the method described in this claim is constructed such that the first means for retaining and securing is a strap, where the strap shares a common edge adjacent to and in communication with the collar, wherein the strap is formed with the collar so as to provide a single-piece condom comprising: the retaining strap formed of a common stretchable material with the collar, and elongated in the direction of an active male wearer's scrotum, whereby said strap is tightly enclosing the scrotum of the active male wearer, and, wherein the strap is stretched around the testicles of the active male wearer following erection of the penis, thereby inhibiting blood flow away from the penis, which, in turn, causes the erection to be substantially maintained for an extended period of time.

17. The method of claim 12, where the condom to be produced through the method described in this claim is constructed such that the shape-retaining, tubular sheath is rigid enough to accept a non-erect male sex organ into its open, proximal end.

18. The method of claim 12, where the condom to be produced through the method described in this claim is constructed such that the means for retaining within the sexual orifice of a passive participant during intercourse includes the tubular sheath, and said shape-retaining, tubular sheath providing means for preventing the collar at the open proximal end of the device from being pushed into the orifice of a passive participant during intercourse, and wherein said means for retaining the condom within the sexual orifice of a passive participant during intercourse includes a tapered, narrow, neck section of the device having a diameter sufficient to maintain said means for retaining in the vagina or in the anus of a passive user, and the outwardly extending collar comprising means for radially stretching the open end, the collar having a first diameter and a second diameter, said second diameter larger than said first diameter, and the strap integrally formed of and in communication with the peripheral edge of the collar.

19. The method of claim 12, where the condom to be produced through the method described in this claim is additionally comprises a reservoir disposed in a substantially closed compartment inside the closed distal end of the condom, wherein the reservoir consists of the closed distal end and defined by a flange, where the flange is a circumferential piece of the condom, with a hole in the middle, funneled down toward the distal end of the condom, such that when semen is ejaculated into the reservoir the downward direction of the flange is thereby serving to trap the semen in the reservoir and prevent its escape and subsequent backflow.

20. The method of claim 1, where the condom to be produced through the method described in this claim is constructed such that the thick-walled, shape-retaining, tubular sheath, said open proximal end, said closed distal end, said collar, said elastic semi-circular retaining strap, said reservoir or reservoirs at the closed distal end, and the condom having a predetermined length and size, comprising: a first portion comprising a major part of said length of the condom, and said first portion comprising a graduated diameter wherein the tubular sheath is tapered and contoured to more readily be retained by a passive user within a sexual orifice, and a second portion comprising the collar, said first portion, the collar and the strap being co-axial with one another and said second portion being between the first portion and the open end of the second portion, and the second portion and the diameter of the collar are graduated to greater than the diameter of the first portion, the second portion, when in use, providing the strap therein adapted to receive and enclose a wearer's scrotum whereby the strap secures the condom in place upon the erect penis.
